(19) United States
(12) Patent Application Publication
(10) Pub. No.: US 2005/0123602 A1
(43) Pub. Date: Jun. 9, 2005

(54) Rifalazil formulations

(76) Inventor: Arthur F. Michaelis, Devon, PA (US)

Correspondence Address: CLARK & ELBING LLP, 101 FEDERAL STREET, BOSTON, MA, 02110, US

(21) Appl. No.: 10/950,917

(22) Filed: Sep. 27, 2004

Related U.S. Application Data
(60) Provisional application No. 60/506,107, filed on Sep. 25, 2003.

(51) Int. Cl. A61K 9/48; A61K 9/64

(52) U.S. Cl. 424/451; 514/183

(57) ABSTRACT

The invention features pharmaceutical compositions including rifalazil and a micelle-forming excipient and methods of use thereof.

### CROSS-REFERENCE TO RELATED APPLICATIONS

The application claims benefit of U.S. Provisional Application No. 60/506,107, filed Sep. 25, 2003, which is incorporated herein by reference.

### BACKGROUND OF THE INVENTION

The present invention relates to the field of antimicrobial therapy.

Rifalazil, an ansamycin-class antibiotic, has been described in U.S. Pat. No. 4,983,602, where its antibacterial activity has been disclosed.

A microgranulated formulation of rifalazil is disclosed in U.S. Pat. No. 5,547,683. This microgranulated rifalazil was shown to exhibit improved oral bioavailability in comparison to rifalazil crystals, mortar-milled crystals, and suspensions of mortar-milled crystals as determined by the relative AUCs produced for each formulation orally administered to beagles. Phase I clinical trials for rifalazil are described in U.S. Pat. Nos. 6,566,354 and 6,316,433.

A formulation for the oral administration of rifalazil that produces more consistent pharmacokinetics and an enhanced degree of bioavailability among subjects is desirable.

### SUMMARY OF THE INVENTION

We have discovered that the oral bioavailability of rifalazil may be increased to a surprising degree and the coefficient of variation in pharmacokinetic parameters (e.g., Cₘₐₓ and AUC_{∞}) may be decreased to a surprising degree when rifalazil is formulated with a sufficient amount of a micelle-forming excipient.

Accordingly, in one aspect, the invention features a pharmaceutical composition for oral administration in unit dosage form including rifalazil and an amount of micelle-forming excipient sufficient to produce, upon administration to fasted patients, a coefficient of variation in Cₘₐₓ of less than 60%. Desirably, the coefficient of variation in Cₘₐₓ is less than 55%, 50%, 45%, 40%, 35%, 30%, 25%, or even 20%.

The invention also features a pharmaceutical composition for oral administration in unit dosage form including rifalazil and an amount of micelle-forming excipient sufficient to produce, upon administration to fasted patients, a coefficient of variation in AUC₁₂₈ of less than 40%. Desirably, the coefficient of variation in AUC_{∞} is less than 35%, 30%, 25%, or even 20%.

The invention features a pharmaceutical composition for oral administration in unit dosage form including rifalazil and an amount of micelle-forming excipient sufficient to produce, upon administration to fasted patients, a mean bioavailability of greater than 30%. Desirably, the mean bioavailability is greater than 35%, 40%, 45%, or even 50%.

The invention further features a pharmaceutical composition in the form of a liquid-filled capsule suitable for oral administration to a human containing rifalazil and one or more micelle-forming excipients

The liquid-filled capsule may be a hard capsule or a soft capsule. Desirably, the capsule includes a liquid having greater than 20%, 30%, 40%, 50%, 60%, 70%, 80%, or 90% (w/w) micelle-forming excipients.

The liquid-filled capsule may include a hydrophilic polymer to promote the release of rifalazil after administration. Examples of hydrophilic polymers that can be used include, without limitation, polyoxyethylenes and hyaluronic acid. Desirably, the hydrophilic polymer is a polyoxyethylene, such as PEG 300, PEG 400, PEG 600, or combinations thereof.

The liquid-filled capsule of rifalazil can include a gelling agent to promote viscosity. Desirably, the gelling agent is a polyoxyethylene-polyoxypropylene block copolymer. These gelling agents are available under various trade names, including one or more of Synperonic PE series (ICI), Pluronic® series (BASF), Lutrol (BASF), Supronic, Monolan, Pluracare, and Plurodac. The generic term for these copolymers is “poloxamer” (CAS 9003-11-6). These polymers have the formula (I):
HO(C₂H₄O)ₐ(C₃H₆O)_{b}(C₂H₄O)ₐH (I)
where “a” and “b” denote the number of polyoxyethylene and polyoxypropylene units, respectively. These copolymers are available in molecular weights ranging from 1000 to 15000 daltons, and with ethylene oxide/propylene oxide ratios between 0.1 and 0.8 by weight. Formulations of rifalazil according to the invention may include one or more of the polyoxyethylene-polyoxypropylene block copolymers above. Desirably, the gelling agent is Pluronic® F68.

The liquid-filled capsule of rifalazil can include water to prevent dehydration of the capsule. Desirably, the liquid-filled capsule of rifalazil includes between 0.5% and 10%, 0.5% and 8%, 0.5% and 7%, 0.5% and 6%, 0.5% and 5%, 1% and 7%, 2% and 7%, 3% and 7%, or 4% and 6% (w/w) water.

In one embodiment, the liquid-filled capsule contains 65% to 85% (w/w) PEG-35 castor oil and 8% to 25% PEG 400. The liquid-filled capsule may further contain 4% to 6% (w/w) water and, optionally, 0.2% to 1.5% Pluronic® F68.

Particular micelle-forming excipients that may be used in the formulations described herein include polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, sorbitan fatty acid esters, lower alcohol fatty acid esters, and ionic surfactants. Any micelle-forming excipient described herein may be used in the rifalazil formulations of the invention. Desirably, the liquid-filled capsule of rifalazil includes one or more micelle-forming excipients selected from sodium lauryl sulfate, polyoxyl-40 stearate, PEG-3 castor oil, PEG-5 castor oil, PEG-9 castor oil, PEG-16 castor oil, PEG-20 castor oil, PEG-23 castor oil, PEG-30 castor oil, PEG-35 castor oil, PEG-38 castor oil, PEG-40 castor oil, PEG-50 castor oil, PEG-60 castor oil, PEG-100 castor oil, PEG-200 castor oil, PEG-5 hydrogenated castor oil, PEG-7 hydrogenated castor oil, PEG-10 hydrogenated castor oil, PEG-20 hydrogenated castor oil, PEG-25 hydrogenated castor oil, PEG-30 hydrogenated castor oil, PEG-40 hydrogenated castor oil, PEG-45 hydrogenated castor oil, PEG-50 hydrogenated castor oil, PEG-60 hydrogenated castor oil, PEG-80 hydrogenated castor oil, and PEG-100 hydrogenated castor oil.

For any of the above pharmaceutical compositions, the composition can include between 0.01 and 100, 0.1 and 100, 0.1 and 50, 0.1 and 25, 0.1 and 20, 0.1 and 15, 0.1 and 10, 0.1 and 5, or 0.2 and 20 mg of rifalazil. Desirably, the pharmaceutical composition contains about 0.25, 0.5, 0.75, 1.0, 1.5, 2.0, 2.5, 3.0, 3.5, 4.0, 4.5, 5.0, 5.5, 10, 12.5, 15, 20, 25, 30, 35, 40, 45, or 50 mg of rifalazil.

For any of the above pharmaceutical compositions, the composition can include between 20% and 90%, 30% and 90%, 40% and 90%, 50% and 90%, 60% and 90%, or even 70% and 90% (w/w) micelle-forming excipient.

The invention further features a method of treating a bacterial infection in a patient. The method includes the step of administering a unit dosage form including rifalazil and an amount of micelle-forming excipient sufficient to produce, upon administration to fasted patients, a coefficient of variation in Cₘₐₓ of less than 60%, wherein the rifalazil is administered in an amount effective to treat the infection. Desirably, the coefficient of variation in Cₘₐₓ is less than 55%, 50%, 45%, 40%, 35%, 30%, 25%, or even 20%.

The invention also features a method of treating a bacterial infection in a patient that includes the step of administering a unit dosage form including rifalazil and an amount of micelle-forming excipient sufficient to produce, upon administration to fasted patients, a coefficient of variation in AUC_{∞} of less than 40%, wherein the rifalazil is administered in an amount effective to treat the infection. Desirably, the coefficient of variation in AUC_{∞} is less than 35%, 30%, 25%, or even 20%.

The invention further features a method of treating a bacterial infection in a patient that includes the step of administering a unit dosage form including rifalazil and an amount of micelle-forming excipient sufficient to produce, upon administration to fasted patients, a mean bioavailability of greater than 30%, wherein the rifalazil is administered in an amount effective to treat the infection. Desirably, the mean bioavailability is greater than 35%, 40%, 45%, or even 50%.

The invention yet further features a method of treating a bacterial infection in a patient that includes the step of administering a unit dosage form in the form of a liquid-filled capsule including rifalazil and a micelle-forming excipient, wherein the rifalazil is administered in an amount effective to treat the infection.

In any of the above methods, the infection is selected from community-acquired pneumonia, upper and lower respiratory tract infections, skin and soft tissue infections, hospital-acquired lung infections, bone and joint infections, respiratory tract infections, acute bacterial otitis media, bacterial pneumonia, urinary tract infections, complicated infections, noncomplicated infections, pyelonephritis, intra-abdominal infections, deep-seated abcesses, bacterial sepsis, central nervous system infections, bacteremia, wound infections, peritonitis, meningitis, infections after burn, urogenital tract infections, gastro-intestinal tract infections, pelvic inflammatory disease, endocarditis, and other intravascular infections. The methods of treating bacterial infections described herein are also useful in treating an infection is by a Gram-positive bacterium. Desirably, the methods are used to treat infection by a Gram-positive coccus, or by a drug-resistant Gram-positive coccus. Desirably, the Gram-positive coccus is selected from S. aureus, S. epidermidis, S. pneumoniae, S. pyogenes, M. catarrhalis, H. influenzae, and Enterococcus spp.

The methods of the invention can be used to reduce or eliminate the incidence of postoperative infections in patients undergoing surgical procedures or implantation of prosthetic devices.

The invention further features a method of treating an infection by multi-drug resistant bacteria in a patient. The method includes administering to the patient a liquid-filled capsule including rifalazil and a micelle-forming excipient, or any other pharmaceutical composition of the invention, wherein the rifalazil is administered in an amount effective to treat the multi-drug resistant infection. Resistant strains of bacteria include penicillin-resistant, methicillin-resistant, quinolone-resistant, macrolide-resistant, and/or vancomycin-resistant bacterial strains. The multi-drug resistant bacterial infections to be treated using the methods of the invention include, for example, infections by penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

The invention also features a method of treating or preventing the development of an atherosclerosis-associated disease in a patient. The method includes administering to the patient a liquid-filled capsule including rifalazil and a micelle-forming excipient, or any other pharmaceutical composition of the invention, wherein the rifalazil is administered in an amount effective to treat or prevent the development of the atherosclerosis-associated disease in the patient. The patient is typically diagnosed as having the atherosclerosis-associated disease (or being at increased risk of developing the disease) or as having macrophages or foam cells infected with C. pneumoniae prior to the administration of a liquid-filled rifalazil capsule.

The invention also features a method of reducing the level of C-reactive protein in a patient in need thereof. This method includes administering to the patient a liquid-filled capsule including rifalazil and a micelle-forming excipient, or any other pharmaceutical composition of the invention, wherein the rifalazil is administered in an amount effective to reduce the level of C-reactive protein in the patient. In one embodiment, the patient has not been diagnosed as having a bacterial infection. In another embodiment, the patient has been diagnosed as having macrophages or foam cells infected with C. pneumoniae.

The invention also features a method for reducing C. pneumoniae replication in macrophages or foam cells in a patient in need thereof. This method includes administering to the patient a liquid-filled capsule including rifalazil and a micelle-forming excipient, or any other pharmaceutical composition of the invention, wherein the rifalazil is administered in an amount effective to reduce C. pneumoniae replication in macrophages or foam cells in the patient.

The invention also features a method for treating a persistent C. pneumoniae infection in macrophages or foam cells in a patient. The method includes administering to the patient a liquid-filled capsule including rifalazil and a micelle-forming excipient, or any other pharmaceutical composition of the invention, wherein the rifalazil is administered in an amount effective to treat the C. pneumoniae infection in macrophages or foam cells in the patient.

The invention also features a method for treating a chronic disease associated with an infection of C. pneumoniae. This method includes the step of administering to the patient a liquid-filled capsule including rifalazil and a micelle-forming excipient, or any other pharmaceutical composition of the invention, wherein the rifalazil is administered in an amount effective to treat the infection.

The invention features a method for treating a patient diagnosed as being infected with a bacterium having a multiplying form and a non-multiplying form by administering to the patient (i) a liquid-filled capsule including rifalazil and a micelle-forming excipient, or any other pharmaceutical composition of the invention, and (ii) a second antibiotic that is effective against the multiplying form of the bacterium, wherein the two antibiotics are administered in amounts and for a duration that together are effective to treat the infection.

In one preferred method of carrying out the foregoing method, the antibiotic that is effective against the multiplying form of the bacterium is administered in an amount and for a duration effective to reduce the number of bacteria in the patient to less than about 10⁶ organisms/mL. This typically takes from a few hours to 1, 2, or 3 days, but may take as long as a week. After this has been achieved, the patient is then administered a liquid-filled capsule including rifalazil and a micelle-forming excipient, wherein the rifalazil is administered in an amount and for a duration effective to complete the treatment of the patient. Antibiotics that are effective against the multiplying form of the bacterium include any of the antibiotics described herein.

The invention also features a method of treating a patient diagnosed as having a chronic disease associated with a bacterial infection caused by bacteria capable of establishing a cryptic phase. The method includes the step of administering to the patient a liquid-filled capsule including rifalazil and a micelle-forming excipient, or any other pharmaceutical composition of the invention, wherein the rifalazil is administered in an amount effective to treat the patient.

The invention features a method of treating the cryptic phase of a bacterial infection. This method includes the step of administering to the patient a liquid-filled capsule including rifalazil and a micelle-forming excipient, or any other pharmaceutical composition of the invention. The administering is for a time and in an amount effective to treat the cryptic phase of the bacterial infection.

The invention features a method of treating a bacterial infection in a patient by (a) treating the multiplying form of the bacteria by administering an antibiotic to the patient for a time and an amount sufficient to treat the multiplying form, and (b) treating the non-multiplying form of the bacteria by administering a liquid-filled capsule including rifalazil and a micelle-forming excipient, or any other pharmaceutical composition of the invention, wherein the administering is for a time and in an amount effective to treat the non-multiplying form.

Preferably, the bacterial infection is caused by one of the following: Chlamydia spp. (e.g., C. trachomatis, C. pneumoniae, C. psittaci, C. suis, C. pecorum, C. abortus, C. caviae, C. felis, C. muridarum), N. hartmannellae, W. chondrophila, S. negevensis, or P. acanthamoeba.

The time effective to treat a cryptic phase or other non-multiplying form of a bacterium ranges from one day to one year. In certain instances, treatment can be for several weeks or months, or even extended over the lifetime of the individual patient, if necessary. For example, the duration of treatment may be at least 30 days, at least 45 days, at least 90 days, or at least 180 days. Ultimately, it is most desirable to extend the treatment for such a time that the non-multiplying form is no longer detectable.

The invention also features a method for treating a patient having antibiotic-associated bacterial diarrhea or an infection of C. difficile, or preventing the disease or infection in the patient. The method includes the step of administering to the patient a liquid-filled capsule including rifalazil and a micelle-forming excipient, or any other pharmaceutical composition of the invention, wherein the rifalazil is administered in an amount effective to treat the infection. The method may be employed as an initial treatment of a patient having or being at risk for developing antibiotic-associated bacterial diarrhea or infection of C. difficile, or it may be employed to treat patients for whom the initial treatment (e.g., with metronidazole or vancomycin) has failed to fully treat the antibiotic-associated bacterial diarrhea or an infection of C. difficile. The method may be employed, for example, when the patient is colonized with C. difficile organisms that are resistant to one or more of metronidazole, vancomycin, and rifampicin.

For any of the methods described herein, rifalazil may be administered in conjunction with one or more additional agents such as anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) and steroids (e.g., cortisone, dexamethasone, hydrocortisone, methylprednisolone, prednisolone, prednisone, triamcinolone)), antibacterial agents (e.g., aminoglycosides, amphenicols, ansamycins, β-Lactams, carbapenems, cephalosporins, cephamycins, lincosamides, macrolides, polypeptides, tetracyclines, 2,4-diaminopyrimidines, nitrofurans, quinolones, sulfonamides, lipopeptides, oxazolidones, ketolides, or sulfones. Exemplary antibiotics include amikacin, gentamicin, kanamycin, tetracycline, vancomycin, teicoplanin, azithromycin, clarithromycin, erythromycin, gatifloxacin, levofloxacin, amoxicillin, and metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), or lipid lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These additional agents may be administered within 14 days, 7 days, 1 day, 12 hours, or 1 hour of the administration of a liquid-filled rifalazil capsule, or simultaneously therewith. The additional therapeutic agents may be present in the same or different pharmaceutical compositions as the liquid-filled rifalazil capsule. When present in different pharmaceutical compositions, different routes of administration may be used. For example, a second agent may be administered orally or by intramuscular or subcutaneous injection. Agents that can be administered in conjunction with rifalazil include any of the agents described herein.

In any of the above methods, rifalazil can be administered in a unit dosage form including rifalazil and a micelle-forming excipient. The micelle-forming excipient is present in an amount sufficient to produce, upon administration to fasted patients, a coefficient of variation in Cₘₐₓ of less than 60%, a coefficient of variation in AUC_{∞} of less than 40%, and/or a mean bioavailability of greater than 30%.

The invention features a method of reducing the food effect exhibited by rifalazil administered to a patient. The method includes the steps of: (i) mixing rifalazil with a micelle-forming excipient; and (ii) administering the mixture of step (i) to the patient, wherein the mixture includes between 20% and 90% (w/w) micelle forming excipient. Desirably, the mixture includes 30% and 90%, 40% and 90%, 50% and 90%, 60% and 90%, or even 70% and 90% (w/w) micelle-forming excipient.

The invention further features a method of increasing the oral bioavailability of rifalazil administered to a patient. The method includes the steps of: (i) mixing rifalazil with a micelle-forming excipient; and (ii) administering the mixture of step (i) to the patient, wherein the mixture includes 20% to 90% (w/w) micelle forming excipient. Desirably, the mixture includes 30% and 90%, 40% and 90%, 50% and 90%, 60% and 90%, or even 70% and 90% (w/w) micelle-forming excipient.

The methods and compositions described herein can also be used to generate information useful, for example, for increasing investment in a company or increasing consumer demand for the methods and/or compositions.

The invention therefore features a method of increasing consumer demand for a pharmaceutical composition or therapeutic regimen described herein. The method includes the step of disseminating information about the pharmaceutical composition or therapeutic regimen.

The invention further features a method of increasing investment in a company seeking governmental approval for the sale of a pharmaceutical composition or therapeutic regimen described herein. The method includes the steps of i) disseminating information about the pharmaceutical composition or therapeutic regimen and ii) disseminating information about the intent of the company to market the pharmaceutical composition or therapeutic regimen.

Consumer demand for a pharmaceutical composition described herein, optionally with instructions to administer the pharmaceutical composition as part of a regimen described herein, can be increased by disseminating information about the utility, efficacy, or safety of the pharmaceutical composition or therapeutic regimen. Consumers include health maintenance organizations, hospitals, doctors, and patients. Typically, the information will be disseminated prior to a governmental approval for the sale of a composition or therapeutic regimen of the invention.

A company planning to sell a pharmaceutical composition described herein, optionally with instructions to administer the pharmaceutical composition as part of a regimen described herein, can increase investment therein by disseminating information about the company's intention to seek governmental approval for the sale of and disseminating information about the pharmaceutical composition or therapeutic regimen. For example, the company can increase investment by disseminating information about in vivo studies conducted, or planned, by the company, including, without limitation, information about the toxicity, efficacy, or dosing requirements of a pharmaceutical composition or therapeutic regimen of the invention. The company can also increase investment by disseminating information about the projected date of governmental approval of a pharmaceutical composition or therapeutic regimen of the invention.

Information can be disseminated in any of a variety of ways, including, without limitation, by press release, public presentation (e.g., an oral or poster presentation at a trade show or convention), on-line posting at a web site, and mailing. Information about the pharmaceutical composition or therapeutic regimen can include, without limitation, a structure, diagram, figure, chemical name, common name, tradename, formula, reference label, or any other identifier that conveys the identity of the pharmaceutical composition or therapeutic regimen of the invention to a person.

By “in vivo studies” is meant any study in which a pharmaceutical composition or therapeutic regimen of the invention is administered to a mammal, including, without limitation, non-clinical studies, e.g., to collect data concerning toxicity and efficacy, and clinical studies.

By “projected date of governmental approval” is meant any estimate of the date on which a company will receive approval from a governmental agency to sell, e.g., to patients, doctors, or hospitals, a pharmaceutical composition or therapeutic regimen of the invention. A governmental approval includes, for example, the approval of a drug application by the Food and Drug Administration, among others.

As used herein, “bioavailability” refers to the fraction of drug absorbed following oral administration to a patient. Under fasted conditions the bioavailability of rifalazil formulated as described herein is at least 25%, but may be greater than 30%, 35%, 40%, 45%, or even 50% of the dose administered.

By “coefficient of variation” is meant is the arithmetic standard deviation divided by the arithmetic mean for a particular pharmacokinetic parameter, wherein the data is obtained from a pharmacokinetic study involving 12 or more patients.

By “Cₘₐₓ” is meant the maximum concentration of rifalazil achieved in the blood after dosing.

By “AUC_{∞}” is meant the integrated area under the rifalazil plasma concentration versus time curve from t=0 to ∞.

By “food effect” is meant a difference between mean pharmacokinetic parameters Cₘₐₓ, Tₘₐₓ, AUC_{∞}, and bioavailability for rifalazil administered under fasted conditions in comparison to rifalazil administered under fed conditions.

As used herein, “reducing the food effect” refers to narrowing the difference between any one of Cₘₐₓ, Tₘₐₓ, AUC_{∞}, and bioavailability for rifalazil administered under fasted conditions in comparison to rifalazil administered under fed conditions, such that the differences are less than those observed for microgranulated rifalazil.

By “fed” or “fed conditions” is meant a subject has eaten within 30 minutes prior to drug administration.

By “fasted” or “fasted conditions” is meant a subject has not eaten for twelve hours prior and four hours subsequent to drug administration.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve or stabilize the patient's condition. Thus, in the claims and embodiments, treating is the administration to a patient either for therapeutic or prophylactic purposes.

By “patient” is meant a human.

As used herein, the term “administration” or “administering” refers to peroral administration of rifalazil to a patient.

As used herein, “an amount sufficient” refers to an amount of micelle-forming excipient in a unit dosage formulation of rifalizil necessary to decrease the coefficient of variation in Cₘₐₓ, decrease the coefficient of variation in AUC_{∞}, reduce the food effect, or increase bioavailability in comparison to microgranulated rifalazil. The sufficient amount of micelle-forming excipient used to practice the invention varies depending upon the amount of rifalazil in the unit dosage formulation and the nature of the micelle-forming excipient. The sufficient amount can be determined by performing pharmacokinetic studies as described in Example 6.

By “effective” amount is meant the amount of rifalazil required to treat or prevent an infection or a disease associated with an infection. The effective amount of rifalazil used to practice the invention for therapeutic or prophylactic treatment of conditions caused by or contributed to by a microbial infection varies depending upon the manner of administration, the age, body weight, and general health of the subject. Ultimately, the attending physician will decide the appropriate amount and dosage regimen. Such amount is referred to as an “effective” amount.

The term “unit dosage form” refers to physically discrete units suitable as unitary dosages, such as a pill, tablet, caplet, hard capsule or soft capsule, each unit containing a predetermined quantity of rifalazil. The unit dosage forms of the invention include rifalazil and a micelle-forming excipient.

As used in herein, a “micelle-forming excipient” refers to an excipient capable of forming micelles upon release into gastro-intestinal media. The formation of micelles can be monitored using any of several standard techniques known in the art, including surface tension measurements, solubilization of water insoluble dye, conductivity measurements, and light scattering, among others.

By “hard capsule” is meant a capsule that includes a membrane that forms a two-part, capsule-shaped, container capable of carrying a solid or liquid payload of drug and excipients.

By “soft capsule” is meant a capsule molded into a single container carrying a liquid or semisolid payload of drug and excipients.

By “bacterial infection” is meant the invasion of a host by pathogenic bacteria. For example, the infection may include the excessive growth of bacteria that are normally present in or on the body of a human or growth of bacteria that are not normally present in or on a human. More generally, a bacterial infection can be any situation in which the presence of a bacterial population(s) is damaging to a host body. Thus, a human is “suffering” from a bacterial infection when an excessive amount of a bacterial population is present in or on the person's body, or when the presence of a bacterial population(s) is damaging the cells or other tissue of the person.

By “atherosclerosis” is meant the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the narrowing or obstruction of the blood vessel and the development of atherosclerosis-associated diseases. Atherosclerosis is typically manifested within large and medium-sized arteries, and is often characterized by a state of chronic inflammation within the arteries.

By “atherosclerosis-associated disease” is meant any disorder that is caused by or is associated with atherosclerosis. Typically, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis).

A patient who is being treated for an atherosclerosis-associated disease is one who a medical practitioner has diagnosed as having such a disease. Diagnosis may be by any suitable means. Methods for diagnosing atherosclerosis by measuring systemic inflammatory markers are described, for example, in U.S. Pat. No. 6,040,147, hereby incorporated by reference. Diagnosis and monitoring may employ an electrocardiogram, chest X-ray, echocardiogram, cardiac catheterization, ultrasound (for the measurement of vessel wall thickness), or measurement of blood levels of CPK, CPK-MB, myoglobin, troponin, homocysteine, or C-reactive protein. A patient in whom the development of an atherosclerosis-associated disease is being prevented is one who has not received such a diagnosis. One in the art will understand that these patients may have been subjected to the same tests (electrocardiogram, chest X-ray, etc.) or may have been identified, without examination, as one at high risk due to the presence of one or more risk factors (e.g., family history, hypertension, diabetes mellitus, high cholesterol levels). Thus, prophylactic administration of a liquid-filled rifalazil capsule is considered to be preventing the development of an atherosclerosis-associated disease.

An atherosclerosis-associated disease has been treated or prevented when one or more tests of the disease (e.g., any of those described above) indicate that the patient's condition has improved or the patient's risk reduced. In one example, a reduction in C-reactive protein to normal levels indicates that an atherosclerosis-associated disease has been treated or prevented.

An alternative means by which treatment or prevention is assessed includes determination of the presence of an infection of C. pneumoniae. Any suitable method may be employed (e.g., determination of C. pneumoniae in blood monocytes or in the atheroma itself (e.g., in macrophages or foam cells present in the fatty streak), or detection of C. pneumoniae DNA, RNA, or antibodies to C. pneumoniae in a biological sample from the patient).

“Antibiotic-associated bacterial diarrhea” means the condition wherein antibiotic therapy disturbs the balance of the microbial flora of the gut, allowing pathogenic organisms such as C. difficile to flourish. These organisms cause diarrhea. Antibiotic-associated bacterial diarrhea includes such conditions as C. difficile associated diarrhea (CDAD) and pseudomembranous colitis. When rifalazil is administered as a liquid-filled capsule for the treatment of a C. difficile infection, an effective amount of rifalazil is the amount required to eradicate C. difficile from the patient, or the amount which prevents an infection of C. difficile, as determined by a diagnostic test that detects C. difficile.

“Pseudomembranous colitis,” also known as pseudomembranous enterocolitis or enteritis, means the inflammation of the mucous membrane of both small and large intestine with the formation and passage of pseudomembranous material (composed of fibrin, mucous, necrotic epithelial cells and leukocytes) in the stools.

The term “lower gastrointestinal tract” means the lower part of the small intestine (ileum) and the colon.

By “autoimmune disease” is meant a disease arising from an immune reaction against self-antigens and directed against the individual's own tissues. Examples of autoimmune diseases include but are not limited to systemic lupus erythematosus, rheumatoid arthritis, myasthenia gravis, and Graves' disease.

By “bacteria” is meant a unicellular prokaryotic microorganism that usually multiplies by cell division.

By “bacteria capable of establishing a cryptic phase” is meant any species whose life cycle includes a persistent, non-multiplying phase. These species include but are not limited to C. trachomatis, C. pneumoniae, C. psittaci, C. suis, C. pecorum, C. abortus, C. caviae, C. felis, C. muridarum, N. hartmannellae, W. chondrophila, S. negevensis, and P. acanthamoeba, as well as any other species described in Everett et al. (Int. J. Syst. Evol. Microbiol. 49:415-440 (1999)).

By “chronic disease” is meant an inveterate disease of long continuance, or which progresses slowly, in contrast to an acute disease, which rapidly terminates. A chronic disease may begin with a rapid onset or in a slow insidious manner but it tends to persist for several weeks, months or years, and has a vague and indefinite termination.

By “cryptic phase” is meant the latent or dormant intracellular phase of infection characterized by little or no metabolic activity. The non-replicating cryptic phase is often characteristic of persistent forms of intracellular bacterial infections.

By “elementary body phase” is meant the infectious phase of the bacterial life cycle which is characterized by the presence of elementary bodies (EBs). EBs are small (300-400 nm), infectious, spore-like forms which are metabolically inactive, non-replicating, and found most often in the acellular milieu. EBs possess a rigid outer membrane which protects them from a variety of physical insults such as enzymatic degradation, sonication and osmotic pressure.

By “immunocompromised” is meant a person who exhibits an attenuated or reduced ability to mount a normal cellular or humoral defense to challenge by infectious agents, e.g., viruses, bacterial, fungi, and protozoa. Persons considered immunocompromised include malnourished patients, patients undergoing surgery and bone narrow transplants, patients undergoing chemotherapy or radiotherapy, neutropenic patients, HIV-infected patients, trauma patients, burn patients, patients with chronic or resistant infections such as those resulting from myelodysplastic syndrome, and the elderly, all of who may have weakened immune systems.

By “inflammatory disease” is meant a disease state characterized by (1) alterations in vascular caliber that lead to an increase in blood flow, (2) structural changes in the microvasculature that permit the plasma proteins and leukocytes to leave the circulation, and (3) emigration of the leukocytes from the microcirculation and their accumulation in the focus of injury. The classic signs of acute inflammation are erythema, edema, tenderness (hyperalgesia), and pain. Chronic inflammatory diseases are characterized by infiltration with mononuclear cells (e.g., macrophages, lymphocytes, and plasma cells), tissue destruction, and fibrosis. Non-limiting examples of inflammatory disease include asthma, coronary artery disease, arthritis, conjunctivitis, lymphogranuloma venerum, and salpingitis.

By “intracytoplasmic inclusion” is meant a replicating reticulate body (RB) that has no cell wall. Such inclusions may be detected, for example, through chlamydiae sample isolation and propagation on a mammalian cell lines, followed by fixing and staining using one of a variety of staining methods including Giemsa staining, iodine staining, and immunofluorescence. These inclusions have a typical round or oval appearance.

By “persistent bacterial infection” is meant an infection that is not completely eradicated through standard treatment regimens using antibiotics. Persistent bacterial infections are caused by bacteria capable of establishing a cryptic phase or other non-multiplying form of a bacterium and may be classified as such by culturing bacteria from a patient and demonstrating bacterial survival in vitro in the presence of antibiotics or by determination of anti-bacterial treatment failure in a patient. As used herein, a persistent infection in a patient includes any recurrence of an infection, after receiving antibiotic treatment, from the same species more than two times over the period of two or more years or the detection of the cryptic phase of the infection in the patient. An in vivo persistent infection can be identified through the use of a reverse transcriptase polymerase chain reaction (RT-PCR) to demonstrate the presence of 16S rRNA transcripts in bacterially infected cells after treatment with one or more antibiotics (Antimicrob. Agents Chemother. 12:3288-3297 (2000)).

As used herein, “non-multiplying” phase or bacteria refers to the non-multiplying growth phase of bacteria. Typically, the non-multiplying bacteria will survive standard antimicrobial therapy (see, e.g., Martinez et al., Antimicrob. Agents Chemother. 44:1771-1777 (2000); Riesenfeld et al., Antimicrob. Agents Chemother. 41:2059-2060 (1997); Alonso et al., Microbiology 145:2857-2862 (1999)).

By “replicating phase” is meant the phase of the bacterial cell cycle characterized by the presence of an RB. The RB is the actively replicating form of the Chlamydia. It contains no cell wall and is detected as an inclusion in the cell.

The term “microbial infection” refers to the invasion of the host patient by pathogenic microbes. This includes the excessive growth of microbes that are normally present in or on the body of a patient. More generally, a microbial infection can be any situation in which the presence of a microbial population(s) is damaging to a host patient. Thus, a patient is “suffering” from a microbial infection when excessive numbers of a microbial population are present in or on a patient's body, or when the presence of a microbial population(s) is damaging the cells or other tissue of a patient.

When administered to a human, rifalazil formulations described herein provide an increase in the bioavailability of rifalazil in comparison to the administration of microgranulated rifalazil disclosed in U.S. Pat. No. 5,547,683. The rifalazil formulations also decrease the coefficient of variation in pharmacokinetic parameters (e.g., Cₘₐₓ and AUC_{∞}) in comparison to the microgranulated formulation.

### BRIEF DESCRIPTION OF THE DRAWINGS

FIG. 1 is a graph depicting the dissolution rates of rifalazil from liquid-filled hard capsules in acidic media, simulated intestinal media, and water.

FIG. 2 is a graph depicting the dissolution rate of rifalazil from microgranular powder-filled hard capsules in acidic media.

FIG. 3 is a graph depicting the mean plasma rifalazil concentrations in dogs, fed or fasted, upon administration (PO) of liquid-filled capsules or microgranulated-filled capsules of rifalazil.

### DETAILED DESCRIPTION

The invention provides pharmaceutical formulations including rifalazil and a micelle-forming excipient in an amount sufficient to alter the pharmacokinetics of rifalazil, e.g., by decreasing the coefficient of variation in Cₘₐₓ, decreasing the coefficient of variation in AUC_{∞}, reducing the food effect, and/or increasing the bioavailability of rifalazil in comparison to the microgranulated formulation of rifalazil.

Formulation

As described herein, micelle-forming excipients can be added to rifalazil in a unit dosage form for oral administration. The excipients likely promote the solubilization of rifalazil in the gut, enhancing absorption and enhancing the uniformity of the bioavailability of rifalazil. The excipients used are restricted to those that have a high degree of safety in humans.

A variety of micelle-forming excipients may be used for the formulation of rifalazil including those disclosed in U.S. Pat. No. 6,365,637, incorporated herein by reference and compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, sorbitan fatty acid esters, lower alcohol fatty acid esters, and ionic surfactants. Commercially available examples for each class of excipient are provided below.

Polyethoxylated fatty acids may be used as excipients for the formulation of rifalazil. Examples of commercially available polyethoxylated fatty acid monoester surfactants include: PEG 4-100 monolaurate (Crodet L series, Croda), PEG 4-100 monooleate (Crodet O series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-1EX, Nikko, and Coster K1, Condea), PEG-2 stearate (Nikkol MYS-2, Nikko), PEG-2 oleate (Nikkol MYO-2, Nikko), PEG-4 laurate (Mapeg® 200 ML, PPG), PEG-4 oleate (Mapeg® 200 MO, PPG), PEG-4 stearate (Kessco® PEG 200 MS, Stepan), PEG-5 stearate (Nikkol TMGS-5, Nikko), PEG-5 oleate (Nikkol TMGO-5, Nikko), PEG-6 oleate (Algon OL 60, Auschem SpA), PEG-7 oleate (Algon OL 70, Auschem SpA), PEG-6 laurate (Kessco® PEG300 ML, Stepan), PEG-7 laurate (Lauridac 7, Condea), PEG-6 stearate (Kessco® PEG300 MS, Stepan), PEG-8 laurate (Mapeg® 400 ML, PPG), PEG-8 oleate (Mapeg® 400 MO, PPG), PEG-8 stearate (Mapeg® 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco® PEG 600 ML, Stepan), PEG-12 oleate (Kessco® PEG 600 MO, Stepan), PEG-12 ricinoleate (CAS# 9004-97-1), PEG-12 stearate (Mapeg® 600 MS, PPG), PEG-15 stearate (Nikkol TMGS-15, Nikko), PEG-15 oleate (Nikkol TMGO-15, Nikko), PEG-20 laurate (Kessco® PEG 1000 ML, Stepan), PEG-20 oleate (Kessco® PEG 1000 MO, Stepan), PEG-20 stearate (Mapeg® 1000 MS, PPG), PEG-25 stearate (Nikkol MYS-25, Nikko), PEG-32 laurate (Kessco® PEG 1540 ML, Stepan), PEG-32 oleate (Kessco® PEG 1540 MO, Stepan), PEG-32 stearate (Kessco® PEG 1540 MS, Stepan), PEG-30 stearate (Myrj 51), PEG-40 laurate (Crodet L40, Croda), PEG-40 oleate (Crodet O40, Croda), PEG-40 stearate (Emerest® 2715, Henkel), PEG-45 stearate (Nikkol MYS-45, Nikko), PEG-50 stearate (Myrj 53), PEG-55 stearate (Nikkol MYS-55, Nikko), PEG-100 oleate (Crodet O-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf.). Formulations of rifalazil according to the invention may include one or more of the polyethoxylated fatty acids above.

Polyethylene glycol fatty acid diesters may also be used as excipients for the formulation of rifalazil. Examples of commercially available polyethylene glycol fatty acid diesters include: PEG-4 dilaurate (Mapeg® 200 DL, PPG), PEG-4 dioleate (Mapeg® 200 DO, PPG), PEG-4 distearate (Kessco® 200 DS, Stepan), PEG-6 dilaurate (Kessco® PEG 300 DL, Stepan), PEG-6 dioleate (Kessco® PEG 300 DO, Stepan), PEG-6 distearate (Kessco® PEG 300 DS, Stepan), PEG-8 dilaurate (Mapeg® 400 DL, PPG), PEG-8 dioleate (Mapeg® 400 DO, PPG), PEG-8 distearate (Mapeg® 400 DS, PPG), PEG-10 dipalmitate (Polyaldo 2PKFG), PEG-12 dilaurate (Kessco® PEG 600 DL, Stepan), PEG-12 distearate (Kessco® PEG 600 DS, Stepan), PEG-12 dioleate (Mapeg® 600 DO, PPG), PEG-20 dilaurate (Kessco® PEG 1000 DL, Stepan), PEG-20 dioleate (Kessco® PEG 1000 DO, Stepan), PEG-20 distearate (Kessco® PEG 1000 DS, Stepan), PEG-32 dilaurate (Kessco® PEG 1540 DL, Stepan), PEG-32 dioleate (Kessco® PEG 1540 DO, Stepan), PEG-32 distearate (Kessco® PEG 1540 DS, Stepan), PEG-400 dioleate (Cithrol 4DO series, Croda), and PEG-400 distearate Cithrol 4DS series, Croda). Formulations of rifalazil according to the invention may include one or more of the polyethylene glycol fatty acid diesters above.

PEG-fatty acid mono- and di-ester mixtures may be used as excipients for the formulation of rifalazil. Examples of commercially available PEG-fatty acid mono- and di-ester mixtures include: PEG 4-150 mono, dilaurate (Kessco® PEG 200-6000 mono, Dilaurate, Stepan), PEG 4-150 mono, dioleate (Kessco® PEG 200-6000 mono, Dioleate, Stepan), and PEG 4-150 mono, distearate (Kessco® 200-6000 mono, Distearate, Stepan). Formulations of rifalazil according to the invention may include one or more of the PEG-fatty acid mono- and di-ester mixtures above.

In addition, polyethylene glycol glycerol fatty acid esters may be used as excipients for the formulation of rifalazil. Examples of commercially available polyethylene glycol glycerol fatty acid esters include: PEG-20 glyceryl laurate (Tagat® L, Goldschmidt), PEG-30 glyceryl laurate (Tagat® L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldo® MS-20 KFG, Lonza), PEG-20 glyceryl oleate (Tagat® O, Goldschmidt), and PEG-30 glyceryl oleate (Tagat® O2, Goldschmidt). Formulations of rifalazil according to the invention may include one or more of the polyethylene glycol glycerol fatty acid esters above.

Alcohol-oil transesterification products may also be used as excipients for the formulation of rifalazil. Examples of commercially available alcohol-oil transesterification products include: PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PRT 56, Pulcra SA), PEG-60 castor oil (Nikkol CO-60TX, Nikko), PEG-100 castor oil, PEG-200 castor oil (Eumulgin® PRT 200, Pulcra SA), PEG-5 hydrogenated castor oil (Nikkol HCO-5, Nikko), PEG-7 hydrogenated castor oil (Cremophor WO7, BASF), PEG-10 hydrogenated castor oil (Nikkol HCO-10, Nikko), PEG-20 hydrogenated castor oil (Nikkol HCO-20, Nikko), PEG-25 hydrogenated castor oil (Simulsol® 1292, Seppic), PEG-30 hydrogenated castor oil (Nikkol HCO-30, Nikko), PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO-100, Nikko), PEG-6 corn oil (Labrafil® M 2125 CS, Gattefosse), PEG-6 almond oil (Labrafil® M 1966 CS, Gattefosse), PEG-6 apricot kernel oil (Labrafil® M 1944 CS, Gattefosse), PEG-6 olive oil (Labrafil® M 1980 CS, Gattefosse), PEG-6 peanut oil (Labrafil® M 1969 CS, Gattefosse), PEG-6 hydrogenated palm kernel oil (Labrafil® M 2130 BS, Gattefosse), PEG-6 palm kernel oil (Labrafil® M 2130 CS, Gattefosse), PEG-6 triolein (Labrafil® M 2735 CS, Gattefosse), PEG-8 corn oil (Labrafil® WL 2609 BS, Gattefosse), PEG-20 corn glycerides (Crovol M40, Croda), PEG-20 almond glycerides (Crovol A40, Croda), PEG-25 trioleate (TAGAT® TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labrafac® Hydro, Gattefosse), PEG-8 caprylic/capric glycerides (Labrasol, Gattefosse), PEG-6 caprylic/capric glycerides (SOFTIGEN®767, Huls), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol DS, Taiwan Surf.), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nikkol Pentarate 408, Nikko). Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants. Formulations of rifalazil according to the invention may include one or more of the alcohol-oil transesterification products above.

Polyglycerized fatty acids may also be used as excipients for the formulation of rifalazil. Examples of commercially available polyglycerized fatty acids include: polyglyceryl-2 stearate (Nikkol DGMS, Nikko), polyglyceryl-2 oleate (Nikkol DGMO, Nikko), polyglyceryl-2 isostearate (Nikkol DGMIS, Nikko), polyglyceryl-3 oleate (Caprol® 3GO, ABITEC), polyglyceryl-4 oleate (Nikkol Tetraglyn 1-O, Nikko), polyglyceryl-4 stearate (Nikkol Tetraglyn 1-S, Nikko), polyglyceryl-6 oleate (Drewpol 6-1-O, Stepan), polyglyceryl-10 laurate (Nikkol Decaglyn 1-L, Nikko), polyglyceryl-10 oleate (Nikkol Decaglyn 1-O, Nikko), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-O, Nikko), polyglyceryl-3 dioleate (Cremophor GO32, BASF), polyglyceryl-3 distearate (Cremophor GS32, BASF), polyglyceryl-4 pentaoleate (Nikkol Tetraglyn 5-O, Nikko), polyglyceryl-6 dioleate (Caprol® 6G20, ABITEC), polyglyceryl-2 dioleate (Nikkol DGDO, Nikko), polyglyceryl-10 trioleate (Nikkol Decaglyn 3-O, Nikko), polyglyceryl-10 pentaoleate (Nikkol Decaglyn 5-O, Nikko), polyglyceryl-10 septaoleate (Nikkol Decaglyn 7-O, Nikko), polyglyceryl-10 tetraoleate (Caprol® 10G40, ABITEC), polyglyceryl-10 decaisostearate (Nikkol Decaglyn 10-IS, Nikko), polyglyceryl-101 decaoleate (Drewpol 10-10-O, Stepan), polyglyceryl-10 mono, dioleate (Caprol® PGE 860, ABITEC), and polyglyceryl polyricinoleate (Polymuls, Henkel). Formulations of rifalazil according to the invention may include one or more of the polyglycerized fatty acids above.

In addition, propylene glycol fatty acid esters may be used as excipients for the formulation of rifalazil. Examples of commercially available propylene glycol fatty acid esters include: propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-O6, Eastman), propylene glycol dicaprylate dicaprate (Captex® 200, ABITEC), propylene glycol dioctanoate (Captex®, ABITEC), propylene glycol caprylate caprate (LABRAFAC PG, Gattefosse), propylene glycol dilaurate, propylene glycol distearate (Kessco® PGDS, Stepan), propylene glycol dicaprylate (Nikkol Sefsol 228, Nikko), and propylene glycol dicaprate (Nikkol PDD, Nikko). Formulations of rifalazil according to the invention may include one or more of the propylene glycol fatty acid esters above.

Mixtures of propylene glycol esters and glycerol esters may also be used as excipients for the formulation of rifalazil. One preferred mixture is composed of the oleic acid esters of propylene glycol and glycerol (Arlacel 186). Examples of these surfactants include: oleic (ATMOS 300, ARLACEL 186, ICI), and stearic (ATMOS 150). Formulations of rifalazil according to the invention may include one or more of the mixtures of propylene glycol esters and glycerol esters above.

Furthermore, mono- and diglycerides may be used as excipients for the formulation of rifalazil. Examples of commercially available mono- and diglycerides include: monopalmitolein (C16:1) (Larodan), monoelaidin (C18:1) (Larodan), monocaproin (C6) (Larodan), monocaprylin (Larodan), monocaprin (Larodan), monolaurin (Larodan), glyceryl monomyristate (C14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen® 701, Huls), glyceryl monolaurate (ALDO® MLD, Lonza), glycerol monopalmitate (Emalex GMS-P, Nihon), glycerol monostearate (Capmul® GMS, ABITEC), glyceryl mono- and dioleate (Capmul® GMO-K, ABITEC), glyceryl palmitic/stearic (CUTINA MD-A, ESTAGEL-G18), glyceryl acetate (Lamegin® EE, Grunau GmbH), glyceryl laurate (Imwitor® 312, Huls), glyceryl citrate/lactate/oleate/linoleate (Imwitor® 375, Huls), glyceryl caprylate (Imwitor® 308, Huls), glyceryl caprylate/caprate (Capmul® MCM, ABITEC), caprylic acid mono- and diglycerides (Imwitor®988, Huls), caprylic/capric glycerides (Imwitor® 742, Huls), Mono-and diacetylated monoglycerides (Myvacet® 9-45, Eastman), glyceryl monostearate (Aldo® MS, Arlacel 129, ICI), lactic acid esters of mono and diglycerides (LAMEGIN GLP, Henkel), dicaproin (C6) (Larodan), dicaprin (C10) (Larodan), dioctanoin (C8) (Larodan), dimyristin (C14) (Larodan), dipalmitin (C16) (Larodan), distearin (Larodan), glyceryl dilaurate (C12) (Capmul® GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan). Formulations of rifalazil according to the invention may include one or more of the mono- and diglycerides above.

Sterol and sterol derivatives may also be used as excipients for the formulation of rifalazil. Examples of commercially available sterol and sterol derivatives include: cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nikkol BPSH-25, Nikko), PEG-5 soyasterol (Nikkol BPS-5, Nikko), PEG-10 soyasterol (Nikkol BPS-10, Nikko), PEG-20 soyasterol (Nikkol BPS-20, Nikko), and PEG-30 soyasterol (Nikkol BPS-30, Nikko). Formulations of rifalazil according to the invention may include one or more of the sterol and sterol derivatives above.

Polyethylene glycol sorbitan fatty acid esters may also be used as excipients for the formulation of rifalazil. Examples of commercially available polyethylene glycol sorbitan fatty acid esters include: PEG-10 sorbitan laurate (Liposorb L-10, Lipo Chem.), PEG-20 sorbitan monolaurate (Tween® 20, Atlas/ICI), PEG-4 sorbitan monolaurate (Tween® 21, Atlas/ICI), PEG-80 sorbitan monolaurate (Hodag PSML-80, Calgene), PEG-6 sorbitan monolaurate (Nikkol GL-1, Nikko), PEG-20 sorbitan monopalmitate (Tween® 40, Atlas/ICI), PEG-20 sorbitan monostearate (Tween® 60, Atlas/ICI), PEG-4 sorbitan monostearate (Tween® 61, Atlas/ICI), PEG-8 sorbitan monostearate (DACOL MSS, Condea), PEG-6 sorbitan monostearate (Nikkol TS106, Nikko), PEG-20 sorbitan tristearate (Tween® 65, Atlas/ICI), PEG-6 sorbitan tetrastearate (Nikkol GS-6, Nikko), PEG-60 sorbitan tetrastearate (Nikkol GS-460, Nikko), PEG-5 sorbitan monooleate (Tween® 81, Atlas/ICI), PEG-6 sorbitan monooleate (Nikkol TO-106, Nikko), PEG-20 sorbitan monooleate (Tween® 80, Atlas/ICI), PEG-40 sorbitan oleate (Emalex ET 8040, Nihon Emulsion), PEG-20 sorbitan trioleate (Tween® 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween® 120, Atlas/ICI), PEG sorbitol hexaoleate (Atlas G-1086, ICI), polysorbate 80 (Tween® 80, Pharma), polysorbate 85 (Tween® 85, Pharma), polysorbate 20 (Tween® 20, Pharma), polysorbate 40 (Tween® 40, Pharma), polysorbate 60 (Tween® 60, Pharma), and PEG-6 sorbitol hexastearate (Nikkol GS-6, Nikko). Formulations of rifalazil according to the invention may include one or more of the polyethylene glycol sorbitan fatty acid esters above.

In addition, polyethylene glycol alkyl ethers may be used as excipients for the formulation of rifalazil. Examples of commercially available polyethylene glycol alkyl ethers include: PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICI). Formulations of rifalazil according to the invention may include one or more of the polyethylene glycol alkyl ethers above.

Sugar esters may also be used as excipients for the formulation of rifalazil. Examples of commercially available sugar esters include: sucrose distearate (SUCRO ESTER 7, Gattefosse), sucrose distearate/monostearate (SUCRO ESTER 11, Gattefosse), sucrose dipalmitate, sucrose monostearate (Crodesta F- 160, Croda), sucrose monopalmitate (SUCRO ESTER 15, Gattefosse), and sucrose monolaurate (Saccharose monolaurate 1695, Mitsubisbi-Kasei). Formulations of rifalazil according to the invention may include one or more of the sugar esters above.

Polyethylene glycol alkyl phenols are also useful as excipients for the formulation of rifalazil. Examples of commercially available polyethylene glycol alkyl phenols include: PEG-10-100 nonylphenol series (Triton X series, Rohm & Haas) and PEG-15-100 octylphenol ether series (Triton N-series, Rohm & Haas). Formulations of rifalazil according to the invention may include one or more of the polyethylene glycol alkyl phenols above.

Sorbitan fatty acid esters may also be used as excipients for the formulation of rifalazil. Examples of commercially sorbitan fatty acid esters include: sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICI), sorbitan tristearate (Span-65, Atlas/ICI), sorbitan monoisostearate (Crill 6, Croda), and sorbitan sesquistearate (Nikkol SS-15, Nikko). Formulations of rifalazil according to the invention may include one or more of the sorbitan fatty acid esters above.

Esters of lower alcohols (C₂ to C₄) and fatty acids (C₈ to C₁₈) are suitable surfactants for use in the invention. Examples of these surfactants include: ethyl oleate (Crodamol EO, Croda), isopropyl myristate (Crodamol IPM, Croda), isopropyl palmitate (Crodamol IPP, Croda), ethyl linoleate (Nikkol VF-E, Nikko), and isopropyl linoleate (Nikkol VF-IP, Nikko). Formulations of rifalazil according to the invention may include one or more of the lower alcohol fatty acid esters above.

In addition, ionic surfactants may be used as excipients for the formulation of rifalazil. Examples of useful ionic surfactants include: sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of rifalazil according to the invention may include one or more of the ionic surfactants above.

Many of the foregoing excipients are micelle-forming in intestinal media. The formation of micelles can be monitored using any of several standard techniques known in the art, including surface tension measurements, solubilization of water insoluble dye, conductivity measurements, and light scattering, among others. In all of these methods, an abrupt change in some physicochemical property is measured as a function of excipient concentration. The abrupt change occurs when the concentration of excipient is sufficient to form micelles. Above this concentration, also known as the critical micelle concentration (CMC), micelles are present in solution.

Methods for making formulations for oral administration are found, for example, in “Remington: The Science and Practice of Pharmacy” (20th ed., ed. A. R. Gennaro, 2000, Lippincott Williams & Wilkins). Formulations for oral administration (e.g., tablets, pills, caplets, hard capsules, and soft capsules) may, for example, contain any one or combination of the excipients described above along with other excipients as needed. Liquid-filled capsules can include any of the excipients described herein. The capsule will contain from, for example, 0.1 to about 100 mg of rifalazil. Liquid-filled capsules may, for example, contain either solutions or suspensions of rifalazil, depending upon the concentration of rifalazil within the capsule and the excipients used in the formulation.

Rifalazil may be formulated as a pharmaceutically acceptable salt, such as a non-toxic acid addition salt or metal complexe that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid, phosphoric acid, or the like. Metal complexes include zinc, iron, and the like.

Many strategies can be pursued to obtain controlled release in which the rate of release outweighs the rate of metabolism of the therapeutic compound. For example, controlled release can be obtained by the appropriate selection of formulation parameters and ingredients, including, e.g., single or multiple unit capsule compositions, by varying the amount of hydrophilic polymer present in the liquid-filled rifalazil capsule, or by varying the amount of gelling agent in the formulated capsule.

Other Therapeutic Agents

The rifalazil formulations described herein may also include a second therapeutic agent including, for example, another antibiotic, an anesthetic, an antimicrobial agent, a zinc salt, or an anti-inflammatory agent (e.g., an non-steroidal anti-inflammatory or a steroid).

Antibiotics that can be admixed with the liquid-filled rifalazil capsule formulation include: aminoglycosides, such as amikacin, apramycin, arbekacin, bambermycins, butirosin, dibekacin, dihydrostreptomycin, fortimicin(s), fradiomycin, gentamicin, ispamicin, kanamycin, micronomicin, neomycin, neomycin undecylenate, netilmicin, paromomycin, ribostamycin, sisomicin, spectinomycin, streptomycin, streptonicozid, and tobramycin; amphenicols, such as azidamfenicol, chloramphenicol, chloramphenicol palmirate, chloramphenicol pantothenate, florfenicol, and thiamphenicol; ansamycins, such as rifampin, rifabutin, rifapentine, and rifaximin; β-Lactams, such as amidinocillin, amdinocillin, pivoxil, amoxicillin, ampicillin, aspoxicillin, azidocillin, azlocillin, bacampicillin, benzylpenicillinic acid, benzylpenicillin, carbenicillin, carfecillin, carindacillin, clometocillin, cloxacillin, cyclacillin, dicloxacillin, diphenicillin, epicillin, fenbenicillin, floxicillin, hetacillin, lenampicillin, metampicillin, methicillin, mezlocillin, nafcillin, oxacillin, penamecillin, penethamate hydriodide, penicillin G benethamine, penicillin G benzathine, penicillin G benzhydrylamine, penicillin G calcium, penicillin G hydragamine, penicillin G potassium, penicillin G, procaine, penicillin N, penicillin O, penicillin V, penicillin V benzathine, penicillin V hydrabamine, penimepicycline, phenethicillin, piperacillin, pivapicillin, propicillin, quinacillin, sulbenicillin, talampicillin, temocillin and ticarcillin; carbapenems, such as imipenem; cephalosporins, such as 1-carba (dethia) cephalosporin, cefactor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpirimide, cefpodoxime proxetil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin, cephalothin, cefaclor, cefotetan, cefprozil, loracarbef, cefetamet, and cefepime; cephamycins such as cefbuperazone, cefmetazole, cefminox, cefetan, and cefoxitin; monobactams such as aztreonam, carumonam, and tigemonan; oxacephems such as flomoxef and moxolactam; lincosamides such as clindamycin and lincomycin; macrolides such as azithromycin, carbomycin, clarithromycin, erythromycin(s) and derivatives, josamycin, leucomycins, midecamycins, miokamycin, oleandomycin, primycin, rokitamycin, rosaramicin, roxithromycin, spiramycin and troleandomycin; polypeptides such as amphomycin, bacitracin, capreomycin, colistin, enduracidin, enylomycin, fusafungine, gramicidin(s), gramicidin S, mikamycin, polymyxin, polymyxin .β-methanesulfonic acid, pristinamycin, ristocetin, teicoplanin, thiostrepton, tuberactinomycin, tyrocidine, tyrothricin, vancomycin, viomycin(s), virginiamycin and zinc bacitracin; tetracyclines such as spicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin and tetracycline; and 2,4-diaminopyrimidines such as brodimoprim, tetroxoprim and trimethoprim; nitrofurans such as furaltadone, furazolium, nifuradene, nifuratel, nifurfoline, nifurpirinol, nifurprazine, nifurtoinol and nitrofurantoin; quinolones such as amifloxacin, cinoxacin, ciprofloxacin, difloxacin, enoxacin, fleroxacin, flumequine, lomefloxacin, miloxacin, nalidixic acid, norfloxacin, ofloxacin, oxolinic acid, perfloxacin, pipemidic acid, piromidic acid, rosoxacin, temafloxacin, and tosufloxacin; sulfonamides such as acetyl sulfamethoxypyrazine, acetyl sulfisoxazole, azosulfamide, benzylsulfamide, chloramine-β, chloramine-T, dichloramine-T, formosulfathiazole, N₂-formyl-sulfisomidine, N₄-β-D-glucosylsulfanilamide, mafenide, 4′-(methyl-sulfamoyl)sulfanilanilide, p-nitrosulfathiazole, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, sulfanilamidomethanesulfonic acid triethanolamine salt, 4-sulfanilamidosalicyclic acid, N₄-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine and sulfisoxazole; sulfones, such as acedapsone, acediasulfone, acetosulfone, dapsone, diathymosulfone, glucosulfone, solasulfone, succisulfone, sulfanilic acid, p-sulfanilylbenzylamine, p,p′-sulfonyldianiline-N,N′digalactoside, sulfoxone and thiazolsulfone; lipopeptides such as daptomycin; oxazolidones such as linezolid; ketolides such as telithromycin; and miscellaneous antibiotics such as clofoctol, hexedine, magainins, methenamine, methenamine anhydromethylene-citrate, methenamine hippurate, methenamine mandelate, methenamine sulfosalicylate, nitroxoline, squalamine, xibornol, cycloserine, mupirocin, and tuberin.

Preferred non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine, and preferred steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.

Preferred anesthetics include, for example, benzocaine, butamben picrate, tetracaine, dibucaine, prilocaine, etidocaine, mepivacaine, bupivicaine, and lidocaine.

Preferred zinc salts include, for example, zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, and zinc glycinate.

All of the therapeutic agents employed in the pharmaceutical compositions of the invention can be used in the dose ranges currently known and used for these agents. Different concentrations may be employed depending on the clinical condition of the patient, the goal of therapy (treatment or prophylaxis), the anticipated duration, and the severity of the infection or disease for which a liquid-filled rifalazil capsule is being administered. Additional considerations in dose selection include the type of infection, age of the patient (e.g., pediatric, adult, or geriatric), general health, and comorbidity. Determining what concentrations to employ are within the skills of the pharmacist, medicinal chemist, or medical practitioner formulating liquid-filled rifalazil capsule in combination with other therapeutic agents.

Therapy

The pharmaceutical compositions described herein can be used to treat or prevent bacterial infections as well as diseases associated with bacterial infections.

Diseases associated with bacterial infections include, but are not limited to, multiple sclerosis (MS), rheumatoid arthritis (RA), inflammatory bowel disease (IBD), interstitial cystitis (IC), fibromyalgia (FM), autonomic nervous dysfunction (AND, neural-mediated hypotension); pyoderma gangrenosum (PG), chronic fatigue (CF), and chronic fatigue syndrome (CFS).

Several lines of evidence have led to the establishment of a link between bacterial infections and a broad set of inflammatory, autoimmune, and immune deficiency diseases. Thus, the present invention describes methods for treating chronic diseases associated with a persistent infection, such as autoimmune diseases, inflammatory diseases and diseases that occur in immuno-compromised individuals by treating the non-multiplying form of the infection in an individual in need thereof, by administering a rifalazil formulation described herein, or such a rifalazil formulation in conjunction with an antibiotic effective against multiplying bacteria. Progress of the treatment can be evaluated, using the diagnostic tests known in the art, to determine the presence or absence of the bacteria. Physical improvement in the conditions and symptoms typically associated with the disease to be treated can also be evaluated. Based upon these evaluating factors, the physician can maintain or modify the anti-bacterial therapy accordingly.

The therapies described herein can be used for the treatment of chronic immune and autoimmune diseases when patients are demonstrated to have a bacterial infection. These diseases include, but are not limited to, chronic hepatitis, systemic lupus erythematosus, arthritis, thyroidosis, scleroderma, diabetes mellitus, Graves' disease, Beschet's disease, and graft versus host disease (graft rejection). The therapies of this invention can also be used to treat any disorders in which a bacterial infection is a factor or co-factor.

Thus, the present invention can be used to treat a range of disorders in addition to the above immune and autoimmune diseases when demonstrated to be associated with chlamydial infection by the methods of detection described herein; for example, various infections, many of which produce inflammation as primary or secondary symptoms, including, but not limited to, sepsis syndrome, cachexia, circulatory collapse and shock resulting from acute or chronic bacterial infection, acute and chronic parasitic and/or infectious diseases from bacterial, viral or fungal sources, such as a HIV, AIDS (including symptoms of cachexia, autoimmune disorders, AIDS dementia complex and infections) can be treated.

Among the various inflammatory diseases, there are certain features that are generally agreed to be characteristic of the inflammatory process. These include fenestration of the microvasculature, leakage of the elements of blood into the interstitial spaces, and migration of leukocytes into the inflamed tissue. On a macroscopic level, this is usually accompanied by the familiar clinical signs of erythema, edema, tenderness (hyperalgesia), and pain. Inflammatory diseases, such as chronic inflammatory pathologies and vascular inflammatory pathologies, including chronic inflammatory pathologies such as aneurysms, hemorrhoids, sarcoidosis, chronic inflammatory bowel disease, ulcerative colitis, and Crohn's disease and vascular inflammatory pathologies, such as, but not limited to, disseminated intravascular coagulation, atherosclerosis, and Kawasaki's pathology are also suitable for treatment by methods described herein. The invention can also be used to treat inflammatory diseases such as coronary artery disease, hypertension, stroke, asthma, chronic hepatitis, multiple sclerosis, peripheral neuropathy, chronic or recurrent sore throat, laryngitis, tracheobronchitis, chronic vascular headaches (including migraines, cluster headaches and tension headaches) and pneumonia when demonstrated to be pathogenically related to a bacterial infection.

Treatable disorders when associated with a bacterial infection also include, but are not limited to, neurodegenerative diseases, including, but not limited to, demyelinating diseases, such as multiple sclerosis and acute transverse myelitis; extrapyramidal and cerebellar disorders, such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; progressive supranucleo palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and Machado-Joseph)); and systemic disorders (Refsum's disease, abetalipoprotemia, ataxia, telangiectasia, and mitochondrial multi-system disorder); demyelinating core disorders, such as multiple sclerosis, acute transverse myelitis; disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; senile dementia of Lewy body type; Wernicke-Korsakoff syndrome; chronic alcoholism; Creutzfeldt-Jakob disease; subacute sclerosing panencephalitis, Hallerrorden-Spatz disease; and dementia pugilistica.

It is also recognized that malignant pathologies involving tumors or other malignancies, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or mycosis fungoides)); carcinomas (such as colon carcinoma) and metastases thereof; cancer-related angiogenesis; infantile hemangiomas; and alcohol-induced hepatitis. Ocular neovascularization, psoriasis, duodenal ulcers, angiogenesis of the female reproductive tract, can also be treated when demonstrated by the diagnostic procedures described herein to be associated with a bacterial infection.

The following examples are put forth so as to provide those of ordinary skill in the art with a complete disclosure and description of how the methods and compounds claimed herein are performed, made, and evaluated, and are intended to be purely exemplary of the invention and are not intended to be limiting.

### EXAMPLE 1

### Preparation of Liquid-Filled Capsules Containing 1 mg of Rifalazil

PEG-35 castor oil (3,102 g), Pluronic® F68 (44 g), PEG 400 (1,034 g), water (220 g) and rifalazil (6.149 g) were mixed, resulting in a volume of 4.058 L and a rifalazil concentration of 0.66 mL/mg. Capsules (fill weight of 0.66 g and a fill volume of 0.68 mL) were filled with the liquid to produce liquid-filled capsules containing 1 mg of rifalazil each.

### EXAMPLE 2

### Preparation of Liquid-Filled Capsules Containing 2.5 mg of Rifalazil

PEG-35 castor oil (3,102 g), Pluronic® F68 (44 g), PEG 400 (1,034 g), water (220 g) and rifalazil (15.371 g) were mixed, resulting in a volume of 4.058 L and a rifalazil concentration of 0.264 mL/mg. Capsules (fill weight of 0.66 g and a fill volume of 0.68 mL) were filled with the liquid to produce liquid-filled capsules containing 2.5 mg of rifalazil each.

### EXAMPLE 3

### Preparation of Liquid-Filled Capsules Containing 5 mg of Rifalazil

PEG-35 castor oil (3,102 g), Pluronic® F68 (44 g), PEG 400 (1,034 g), water (220 g) and rifalazil (30.743 g) were mixed, resulting in a volume of 4.058 L and a rifalazil concentration of 0.132 mL/mg. Capsules (fill weight of 0.66 g and a fill volume of 0.68 mL) were filled with the liquid to produce liquid-filled capsules containing 5 mg of rifalazil each.

### EXAMPLE 4

### Preparation of Liquid-Filled Capsules Containing 12.5 mg of Rifalazil

PEG-35 castor oil (3,740 g), Pluronic® F68 (44 g), PEG 400 (396 g), water (220 g) and rifalazil (77.519 g) were mixed, resulting in a volume of 4.093 L and a rifalazil concentration of 0.0528 mL/mg. Capsules (fill weight of 0.66 g and a fill volume of 0.68 mL) were filled with the liquid to produce liquid-filled capsules containing 12.5 mg of rifalazil each.

### EXAMPLE 5

### Dissolution Rates in Varying Media

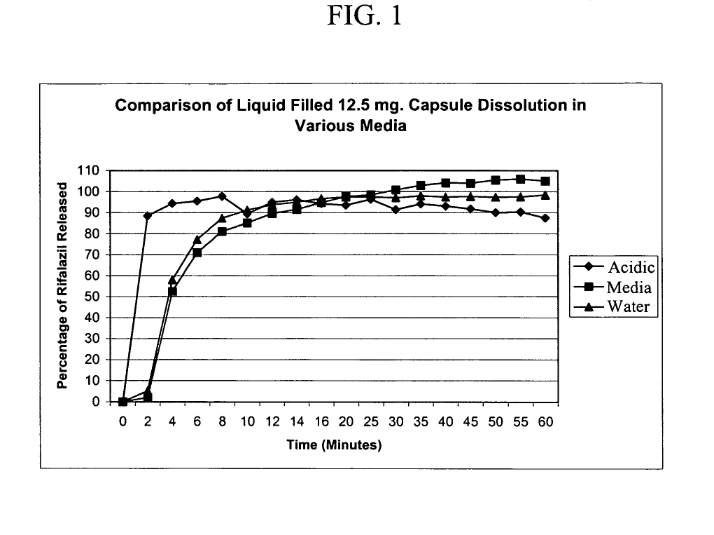

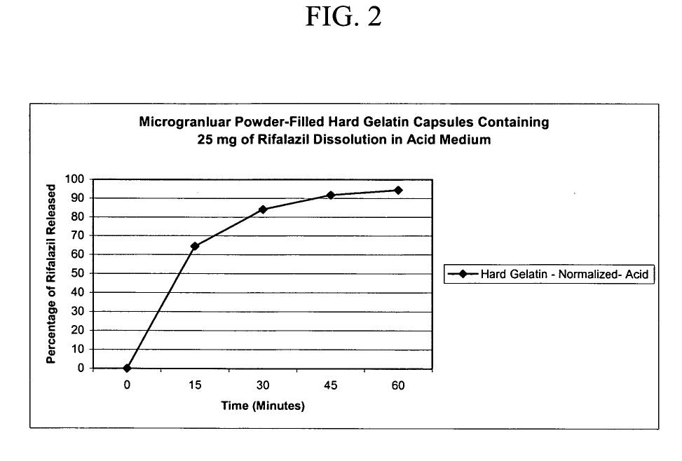

The dissolution rates (mean of three measurements) of rifalazil in a liquid-filled capsule were monitored in different media: acidic media, simulated intestinal media, and water. The resulting data are provided in FIG. 1. The dissolution in intestinal medium and water clearly show the delay caused by the dissolution of the capsule shell. This delay does not occur in the acidic medium using a hard gelatin capsule due to the role of acid in the dissolution of gelatin. However, even in acidic media, which solubilizes rifalazil, the rate of release is much slower for rifalazil using a microgranular powder-filled hard gelatin capsule compared to the liquid filled capsules (compare FIGS. 1 and 2).

### EXAMPLE 6

### Pharmacokinetics of the Liquid-Filled Capsule Underfed and Fasted Conditions

Pharmacokinetic parameters were determined following a single peroral administration of 5 mg of rifalazil in healthy male beagle dogs. The rifalazil was formulated either as a liquid-filled capsule of Example 3 or as a powder-filled capsule containing microgranulated rifalazil as described in U.S. Pat. No. 5,547,683.

Both formulations were administered under fed and fasted conditions. All animals were fasted overnight prior to dosing. Animals designated as “fed” were administered a blended combination of dog chow and water in a 1:3 ratio (e.g. 250 g chow and 750 g water) via oral gavage at a dose volume of 20 mL/kg within approximately 30 minutes prior to dosing and food was provided Ad-libitum after approximately 4 hours following dosing. Animals in “fasted” groups were not fed prior to dosing and food was withheld until after approximately 4 hours after dosing.

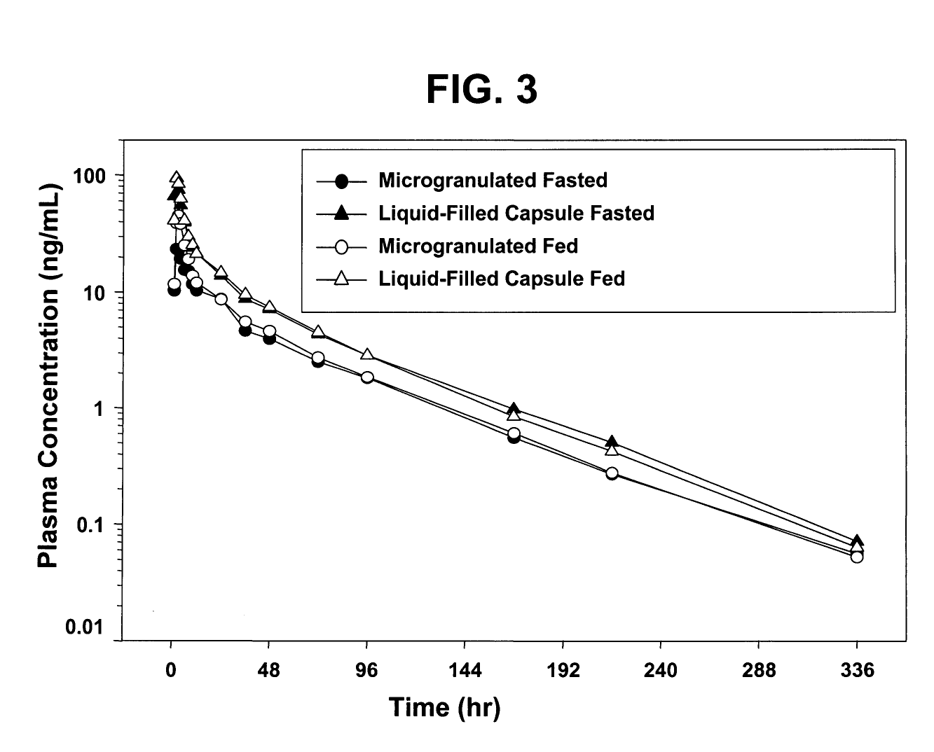

Plasma samples (5.0 mL in EDTA tubes) for determination of rifalazil concentrations in plasma were obtained at hour: 0 (pre-dose) and at hours: 1.0, 2.0, 3.0, 4.0, 6.0, 8.0, 10.0, 12.0, 24, 36, 48, 72, 96, 168, 216 (Day 10), 336 (Day 15), 420, and 504 (Day 21), after administration of the rifalazil in either of the dosage forms. The mean plasma rifalazil concentrations in dogs, fed or fasted, upon administration (PO) of liquid-filled capsules or powder-filled capsules of rifalazil is shown in FIG. 3.

Pharmacokinetic endpoints and parameters were calculated by noncompartmental analysis (NCA) using WinNonlin®.

The pharmacokinetic parameters Tₘₐₓ, Cₘₐₓ, AUCₜ, AUC_{∞}, T_{1/2} (elimination), and F (bioavailability) were calculated as well as the coefficient of variation (CV) in each. The results are provided in Table 1. 100% bioavailability was determined by comparision to the pharmacokinetic profile observed for intravenously administered rifalazil.

TABLE 1

| | Micro- | | Micro- | |
|---|---|---|---|---|
| | granulated | Liquid-filled | granulated | Liquid-filled |
| PK parameter | fasted | fasted | fed | fed |
| Tₘₐₓ (h) | 6.31 ± 7.11 | 1.87 ± 0.33 | 2.69 ± 0.92 | 2.33 ± 0.50 |
| Cₘₐₓ (ng/mL) | 27.2 ± 24.6 | 96.5 ± 18.2 | 52.8 ± 30.5 | 95.8 ± 33.6 |
| AUC_{∞} | 685 ± 359 | 1400 ± 266 | 830 ± 438 | 1420 ± 274 |
| (ng/mL × hr) | | | | |
| T_{1/2} (h) | 52.2 ± 17.3 | 46.8 ± 15.7 | 42.1 ± 14.8 | 43.4 ± 15.8 |
| F (%) | 26 | 53 | 32 | 54 |
| CV Cₘₐₓ (%) | 90.4 | 18.1 | 57.8 | 35.1 |
| CV AUC_{∞} (%) | 53.4 | 19.2 | 52.7 | 19.3 |

The liquid-filled capsules of rifalazil exhibit a surprising increase in Cₘₐₓ under both fed (1.8 fold increase) and fasted (3.5 fold increase) conditions and an increase in AUC_{∞} under both fed (1.7 fold increase) and fasted (2.0 fold increase) conditions in comparison to microgranulated rifalazil.

The liquid-filled capsules of rifalazil also exhibit a surprising increase in bioavailability under both fed (1.7 fold increase) and fasted (2.0 fold increase) conditions in comparison to microgranulated rifalazil.

A comparison of the fed and fasted data obtained for the liquid-filled capsule formulation, i.e., AUC_{∞} (1400 vs. 1420) and Cₘₐₓ (96.5 vs. 95.8), shows no change in PK behavior, e.g., no “food effect.” In contrast, the microgranulated rifalazil exhibits a large food effect as demonstrated by the differences in AUC_{∞} (685 vs. 830) and Cₘₐₓ (27.2 vs. 52.8) under fed and fasted conditions.

A reduction in the coefficient of variation in Cₘₐₓ in both fed (1.6 fold decrease) and fasted (4.8 fold decrease) animals and a reduction in the coefficient of variation in AUC_{∞} in both fed (2.7 fold increase) and fasted (2.7 fold increase) animals is observed for the liquid-filled capsule in comparison to the microgranulated formulation.

Changes in the formulation had no effect upon the elimination half-life (T_{1/2}) of rifalazil.

### OTHER EMBODIMENTS

All publications, patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

1. A pharmaceutical composition for oral administration in unit dosage form comprising rifalazil and an amount of micelle-forming excipient sufficient to produce, upon administration to fasted patients, a coefficient of variation in Cₘₐₓ of less than 60%.

2. A pharmaceutical composition for oral administration in unit dosage form comprising rifalazil and an amount of micelle-forming excipient sufficient to produce, upon administration to fasted patients, a coefficient of variation in AUC_{∞} of less than 40%.

3. A pharmaceutical composition for oral administration in unit dosage form comprising rifalazil and an amount of micelle-forming excipient sufficient to produce, upon administration to fasted patients, a mean bioavailability of greater than 30%.

4. A pharmaceutical composition in the form of a liquid-filled capsule, said capsule comprising rifalazil and a micelle-forming excipient.

5. The pharmaceutical composition of any of claims 1-4, wherein said micelle-forming excipient is selected from polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, sorbitan fatty acid esters, lower alcohol fatty acid esters, and ionic surfactants.

6. The pharmaceutical composition of any of claims 1-4, wherein said micelle-forming excipient is selected from sodium lauryl sulfate, polyoxyl-40 stearate, PEG-3 castor oil, PEG-5, 9, and 16 castor oil, PEG-20 castor oil, PEG-23 castor oil, PEG-30 castor oil, PEG-35 castor oil, PEG-38 castor oil, PEG-40 castor oil, PEG-50 castor oil, PEG-60 castor oil, PEG-100 castor oil, PEG-200 castor oil, PEG-5 hydrogenated castor oil, PEG-7 hydrogenated castor oil, PEG-10 hydrogenated castor oil, PEG-20 hydrogenated castor oil, PEG-25 hydrogenated castor oil, PEG-30 hydrogenated castor oil, PEG-40 hydrogenated castor oil, PEG-45 hydrogenated castor oil, PEG-50 hydrogenated castor oil, PEG-60 hydrogenated castor oil, PEG-80 hydrogenated castor oil, and PEG-100 hydrogenated castor oil.

7. The capsule of claim 4, wherein said micelle-forming excipient PEG-35 castor oil.

8. The capsule of claim 4, further comprising a hydrophilic polymer.

9. The capsule of claim 8, wherein said hydrophilic polymer is selected from PEG 300, PEG 400, and PEG 600.

10. The capsule of claim 4, further comprising a gelling agent.

11. The capsule of claim 10, wherein said gelling agent is a polyoxyethylene-polyoxypropylene block copolymer.

12. The capsule of claim 4, further comprising between 0.5% and 10% (w/w) water.

13. The capsule of claim 4, wherein said capsule comprises a liquid that is 65% to 85% (w/w) PEG-35 castor oil, 8% to 25% PEG 400, 4% to 6% (w/w) water, and 0.2% to 1.5% Pluronic® F68.

14. The capsule of claim 4, wherein said capsule is a hard capsule or a soft capsule.

15. The pharmaceutical composition of any of claims 1-4, said composition comprising between 0.1 and 100 mg of rifalazil.

16. The pharmaceutical composition of claim 15, said composition comprising between 0.1 and 25 mg of rifalazil.

17. The pharmaceutical composition of any of claims 1-4, said composition comprising between 20% and 90% (w/w) micelle-forming excipient.

18. A method of treating a bacterial infection in a patient, said method comprising administering to said patient a unit dosage form comprising rifalazil and an amount of micelle-forming excipient sufficient to produce, upon administration to fasted patients, a coefficient of variation in Cₘₐₓ of less than 60%, wherein said rifalazil is administered in an amount effective to treat said infection.

19. A method of treating a bacterial infection in a patient, said method comprising administering to said patient a unit dosage form comprising rifalazil and an amount of micelle-forming excipient sufficient to produce, upon administration to fasted patients, a coefficient of variation in AUC_{∞} of less than 40%, wherein said rifalazil is administered in an amount effective to treat said infection.

20. A method of treating a bacterial infection in a patient, said method comprising administering to said patient a unit dosage form comprising rifalazil and an amount of micelle-forming excipient sufficient to produce, upon administration to fasted patients, a mean bioavailability of greater than 30%, wherein said rifalazil is administered in an amount effective to treat said infection.

21. A method of treating a bacterial infection in a patient, said method comprising administering to said patient a unit dosage form in the form of a liquid-filled capsule comprising rifalazil and a micelle-forming excipient, wherein said rifalazil is administered in an amount effective to treat said infection.

22. The method of any of claims 18-21, wherein said infection is selected from community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, and intravascular infection.

23. The method of any of claims 18-21, wherein said rifalazil is administered for prophylaxis against an infection resulting from a surgical procedure or implantation of a prosthetic device.

24. The method of any of claims 18-21, wherein said infection is by Gram-positive bacterium.

25. A method of treating an infection by multi-drug resistant bacteria in a patient, said method comprising administering to said patient a liquid-filled capsule comprising rifalazil and a micelle-forming excipient, wherein said rifalazil is administered in an amount effective to treat said infection.

26. A method for reducing Chlamydia pneumoniae replication in macrophages or foam cells in a patient, said method comprising administering to said patient a liquid-filled capsule comprising rifalazil and a micelle-forming excipient, wherein said rifalazil is administered in an amount effective to reduce Chlamydia pneumoniae replication in macrophages or foam cells in said patient.

27. A method of reducing the food effect exhibited by rifalazil administered to a patient, said method comprising:
(i) mixing rifalazil with a micelle-forming excipient; and
(ii) administering the mixture of step (i) to said patient, wherein said mixture comprises between 20% and 90% (w/w) micelle forming excipient.

28. A method of increasing the oral bioavailability of rifalazil administered to a patient, said method comprising:
(i) mixing rifalazil with a micelle-forming excipient; and
(ii) administering the mixture of step (i) to said patient, wherein said mixture comprises between 20% and 90% (w/w) micelle forming excipient.
